(19) United States
(12) Patent Application Publication
(10) Pub. No.: US 2023/0331984 A1
(43) Pub. Date: Oct. 19, 2023

(54) COMPOSITIONS AND METHODS FOR IMPROVING MARINE BIODEGRADABILITY OF POLYMERIC COMPOSITIONS

(71) Applicant: RHODIA BRASIL S.A., São Paulo City (BR)

(72) Inventors: Tarcis Cordeiro Bastos, São Paulo City (BR); Renata Redondo Bonaldi, São Paulo City (BR); Guilherme Lopes Do Lago, São Paulo City (BR); Edson Rodrigues Leme, São Paulo City (BR); Pierre Hansu Pak, São Bernardo do Campo (BR)

(73) Assignee: RHODIA BRASIL S.A., São Paulo City (BR)

(21) Appl. No.: 18/028,096

(22) PCT Filed: Sep. 24, 2020
(86) PCT No.: PCT/IB2020/058931

§ 371 Date: Mar. 23, 2023

(51) Int. Cl. A61K 8/02; A61K 8/88; A61Q 19/00; C08L 77/06; A61K 8/23; A61K 8/25; A61K 8/29; A61K 8/85

(52) U.S. Cl. CPC C08L 77/06; A61K 8/025; A61K 8/0287; A61K 8/23; A61K 8/25; A61K 8/29; A61K 8/85; A61K 8/88; A61Q 19/00; A61K 2800/10; A61K 2800/54

(57) ABSTRACT

The present invention is relative to the use of a composition comprising at least one mineral filler having properties of absorption and/or emission in the far infrared region ranging from wavelength of 2 micrometers to 20 micrometers dispersed in a marine biodegradable polymeric composition for improving marine biodegradability of said marine biodegradable polymeric composition.

The subject of the present invention is a method for improving marine biodegradability of a marine biodegradable polymeric composition, notably used in cosmetic formulations. The present invention is thus related to the use of at least one far infrared mineral filler being dispersed in a marine biodegradable polymeric composition for improving marine biodegradability of the same.

In cosmetic formulations, it is common to use polymeric compositions for various purposes like improving the feel of the cosmetic product; reducing the appearance of wrinkles (by optical scattering or filling the wrinkles); promoting skin exfoliation (as abrasive); and delivering active ingredients to skin (as carriers). Plastics and notably synthetic polymers like polyamides or polyolefins are resistant to biodegradation, which is a factor of water pollution. Nowadays, there is more and more the willingness to reduce the impact of cosmetics on environment and in particular on marine water pollution by using marine biodegradable formulations.

Biopolymers such as aliphatic polyesters and in particular polyhydroxyalkanoates or polylactic acid are widely known for their biodegradability in landfill or composting conditions. Nevertheless, in marine environment some biodegradable polymers do not necessarily biodegrade. It is the case for example of polylactic acid, which does present a very low biodegradability in marine environment.

And, for already marine biodegradable polymeric compositions, there is still a need to further reduce the impact on marine environment by providing innovative solutions which make it possible to go further in marine biodegradability behaviors of existing solutions.

Pursuing its research in this field, the Applicant has now discovered a novel and original approach, which makes it possible to effectively improve marine biodegradability of marine biodegradable polymeric compositions, in particular biodegradable polyesters.

This approach is based on the use of a composition C comprising at least one mineral filler having properties of absorption and/or emission in the far infrared region ranging from wavelength of 2 micrometers to 20 micrometers, said composition C being dispersed in a marine biodegradable polymeric composition.

The resulting marine biodegradable polymeric composition can be then dispersed under the form of particles in the base fluid of a cosmetic formulation and will show improved biodegradability in marine environment. The resulting marine biodegradable polymeric composition can also be spun and used in fiber applications, in particular for industrial or textiles applications.

Indeed, the Applicant has discovered, totally unexpectedly, that the use of dispersed mineral fillers in marine biodegradable polymers like polyhydroxyalkanoates has the effect of boosting the marine biodegradability of the resulting polymeric composition.

When the polymer used is not already marine biodegradable as such, it is possible to previously change it into a marine biodegradable one, by adding specific additives to confer marine biodegradable behavior to the polymeric composition.

The subject of the present invention is therefore the use of a composition C comprising at least one mineral filler M having properties of absorption and/or emission in the far infrared region ranging from wavelength of 2 micrometers to 20 micrometers, said composition C being dispersed in a marine biodegradable polymeric composition, for improving marine biodegradability of said marine biodegradable polymeric composition.

The subject of the present invention is also a method of improving marine biodegradability of a marine biodegradable polymeric composition, comprising the step of dispersing in a marine biodegradable polymeric composition, a composition C comprising at least one mineral filler M having properties of absorption and/or emission in the far infrared region ranging from wavelength of 2 micrometers to 20 micrometers.

It is also an object of the present invention, a marine biodegradable polymeric composition comprising a composition C comprising at least three mineral fillers M of different types having properties of absorption and/or emission in the far infrared region ranging from wavelength of 2 micrometers to 20 micrometers, two of them being selected from the group consisting of oxides, sulfates, carbonates and phosphates and the third one being a silicate, said composition C being dispersed in said polymeric composition, wherein said polymeric composition comprises at least one polyhydroxyalkanoate (PHA), polyglycolic acid (PGA), polycaprolactone (PCL) or polylactic acid (PLA), preferably polyhydroxyalkanoate (PHA), more preferably polyhydroxyalkanoates (PHA) selected in the group consisting of poly-3-hydroxybutyrate (PHB or P3HB), poly(3-hydroxypropionate) (PHP or P3HP), polyhydroxyvalerate (PHV), poly(hydroxybutyrate-hydroxyvalerate (PHBV), Poly(3-hydroxyhexanoate) (PHHx), copolymers thereof and blends thereof and in particular polyhydroxybutyrate (PHB), copolymers thereof and blends thereof.

The subject of the present invention is finally the use of such a specific composition in cosmetic formulations or in industrial or textiles applications.

The term “marine biodegradability” or “biodegradability in marine environment” has to be understood as the aerobic biodegradation of a plastic material when exposed to marine microorganisms of known genera existing in natural seawater, as described in ASTM D6691-01(2017). This test method is designed to index polymer materials that are possibly marine biodegradable, relative to a positive reference material, in an aerobic environment, measuring the total biogas (CO2) produced as a function of time and assessing the degree of marine biodegradability. According to this standard, the reference material that can be cellulose, chitin or Kraft paper. Then, when comparing the tested polymeric composition results to the reference, the marine biodegradability percentage of the polymeric composition can be estimated.

### Marine Biodegradable Polymeric Composition

The invention uses a marine biodegradable polymeric composition.

The term “biodegradable”, when used alone, means that the degradation results from the action of microorganisms such as bacteria, fungi and algae naturally present in the environment (ASTM D883-17 - Standard Terminology Relating to Plastics). Some biodegradable polymers according to ASTM D883-17 can also be marine biodegradable according to ASTM D6691-01(2017) as such, but it is not always the case. In this alternative, it is possible to add to those biodegradable but non-marine biodegradable polymers an additive A that has the ability to bring the marine biodegradability to the polymeric composition containing it.

According to a first embodiment, the polymer is already marine biodegradable as such.

According to a second embodiment, the polymer is not marine biodegradable as such and a marine biodegradability additive A is added to the composition.

In both embodiments, the polymeric composition can be from natural or synthetic origin.

Natural polymers can be those obtained directly from biomass or those produced by natural or genetically modified organisms.

As natural marine biodegradable polymers as such we can cite:
polysaccharides (starches, cellulose, hemicellulose and cellulose derivatives, chitin and some gums); and
polypeptides or proteins (corn zein, wheat gluten, soy protein, collagen, casein, albumin, gelatin, etc).
microbial polyesters, in particular polyhydroxyalkanoates (PHA) like poly-3-hydroxybutyrate (PHB or P3HB), poly(3-hydroxypropionate) (PHP or P3HP), polyhydroxyvalerate (PHV), poly(hydroxybutyrate-hydroxyvalerate (PHBV), Poly(3-hydroxyhexanoate) (PHHx), or poly-ε-caprolactones);
bacterial cellulose;
polyesters synthesized from bio-derived monomers.

As synthetic marine biodegradable polymers as such we can cite:
aliphatic polyesters (polyglycolic acid (PGA), polycaprolactone (PCL), Poly(lactide-co-glycolide) (PLGA);
Poly(vinyl alcohol), and
cellulose esters and derivatives thereof.

Non marine biodegradable polymers as such are for example polyamides (preferably PA66, PA6, PA 5.6, PA6.10, PA10.10 and PA12), polylactic acid (PLA), poly(butylene succinate) (PBS), poly(butylene adipate-co-terephthalate) (PBAT) and poly(vinyl acetate).

The polymer in the biodegradable polymeric composition is preferably selected from the group consisting of polyamides, polyesters, cellulose and derivatives polymers, cellulose esters and derivatives polymers, and derivatives polymers copolymers thereof and blends thereof.

By “cellulose derivatives polymers”, we can cite methyl cellulose (MC), ethyl cellulose (EC), hydroxyethyl cellulose (HEC), hydroxypropyl methylcellulose (HPMC) and carboxymethyl cellulose (CMC).

The term “cellulose esters and derivatives polymers” comprises cellulose esters like cellulose acetate, nitroacetate, formate, propionate and butyrate and derivatives include e.g. cellulose nitrate and ether-esters of cellulose.

The polymer of the biodegradable polymeric composition is preferably already marine biodegradable.

According to one preferential embodiment, the polymer of the marine biodegradable polymeric composition is selected from the group consisting of polyhydroxyalkanoates (PHA), cellulose derivatives polymers, cellulose acetate polymers, polyglycolic acid, polycaprolactone, copolymers thereof and blends thereof.

The polymer of the marine biodegradable polymeric composition is advantageously a polyhydroxyalkanoate (PHA), preferably selected in the group consisting of poly-3-hydroxybutyrate (PHB or P3HB), poly(3-hydroxypropionate) (PHP or P3HP), polyhydroxyvalerate (PHV), poly(hydroxybutyrate-hydroxyvalerate (PHBV), Poly(3-hydroxyhexanoate) (PHHx), copolymers thereof and blends thereof and in particular polyhydroxybutyrate (PHB), copolymers thereof and blends thereof.

### Additive A

When the polymer is not marine biodegradable, typically in the case of polyamide (preferably PA66, PA6, PA 5.6, PA6.10, PA10.10 and PA12), polylactic acid (PLA), poly(butylene succinate) (PBS), poly(butylene adipate-co-terephthalate) (PBAT) and poly(vinyl acetate), a marine biodegradability additive A is advantageously added.

Additive A is typically a composition comprising at least an amorphous carbohydrate-based or starch-based or aromatic-ester modified polymeric material, optionally a plasticizer and water. Examples of suitable additive A compositions are available from BiologiQ, under the tradename ESR (“Eco Starch Resin” or “Eco Sustainable Resin”, like ESR GS-270, GS-300 and GS-330), NuplastiQ and BioSphere ® products and in particular BioSphere®201. Further details on those compositions, methods to produce them, to blend them with the polymeric composition or uses thereof are described in US 2018/0100060 A1, US 2017/0362418 A1 and US 2017/0218184 A1, which are hereby incorporated by reference in their entirety.

The biodegradable polymeric composition is preferably already marine biodegradable.

In this second embodiment, the marine biodegradable polymeric composition comprises:
(a) a polymer selected from the group consisting of polyamide (preferably PA66, PA6, PA 5.6, PA6.10, PA10.10 and PA12), polylactic acid (PLA), poly(butylene succinate) (PBS), poly(butylene adipate-co-terephthalate) (PBAT) and poly(vinyl acetate) copolymers and blends thereof, and
(b) an additive A being a composition comprising :
   (i) at least one carbohydrate-based or starch-based or aromatic-ester modified polymeric material,
   (ii) optionally a plasticizer, and
   (iii) optionally water.

When the polymer is marine biodegradable as such (first embodiment), it is also possible according to another embodiment to add a marine biodegradability additive A as described above.

### Mineral Filler M

According to the invention, the mineral filler(s) M is (are) dispersed in the biodegradable polymeric composition. The term “dispersed” is intended to mean that the mineral fillers are homogeneously incorporated actually into the polymer. In particular, the particles are trapped in the polymer composition. They are not therefore mineral fillers deposited on the polymer, for example in the form of a coating at the surface of the polymer.

Such a dispersion can be obtained by incorporating the mineral filler(s) into the polymer during the synthesis of the latter. One embodiment consists in producing one or more surfactant-stabilized suspension(s) of mineral fillers. The suspension(s) is (are) then added during the synthesis of the polymer.

Said fillers can also be incorporated by mixing the latter with the molten polymer, either directly, or by means of a concentrate of particles in the form of a masterbatch, it being possible for the latter to be subsequently diluted to predetermined concentrations in the polymer mass.

By virtue of such processes, it is possible to obtain polymeric compositions according to the invention, which contain the mineral filler(s) in a manner dispersed in the marine biodegradable polymeric composition.

The mineral filler(s) M used in the present invention have properties of absorption and/or emission in the far infrared region ranging from wavelength of 2 to 20 micrometers. Preferably, the mineral filler(s) M has (have) properties of absorption and/or emission in the far infrared region ranging from wavelength of 3 to 20 micrometers, and even more preferentially from wavelength of 3 to 15 micrometers.

In a preferred embodiment, the at least one mineral filler M is water insoluble.

By “water insoluble” it has to be understood that the solubility is less than 0.1 g per 100 ml of water at 20° C. and 1 atm (US Pharmacopoeia).

The mineral filler(s) M usable according to the invention can be chosen in particular from oxides, sulfates, carbonates, phosphates and silicates.

Preferably, the oxide(s) is (are) chosen from titanium dioxide, silicon dioxide and magnesium oxide.

The sulfate(s) can advantageously be chosen from alkali metal and alkaline-earth metal sulfates, preferably from barium sulfate, calcium sulfate and strontium sulfate.

The carbonate(s) is (are) advantageously chosen from calcium carbonate and sodium carbonate.

Preferably, the silicate(s) is (are) chosen from actinolite, tourmaline, serpentine, kaolinite, montmorillonite, zeolite, micas and zirconium silicate.

The phosphate(s) can be chosen from zirconium phosphates, cerium phosphate, calcium phosphate, sodium phosphate, magnesium phosphate, potassium phosphate, hydroxyapatite and apatite, and mixtures thereof.

In a preferred embodiment, at least one mineral filler M is a silicate, preferably tourmaline.

Preferably, the composition C contains at least two mineral fillers of different types, chosen from the following types: oxides, sulfates, carbonates, phosphates and silicates. It is particularly preferred to have at least one silicate, preferably tourmaline in such an embodiment.

Particularly preferably, the polymeric composition contains at least three mineral fillers of different types, chosen from the above mentioned types. In this case, it is particularly preferred to have at least one silicate, preferably tourmaline in such an embodiment.

According to a first preferred embodiment, the composition C contains at least two mineral fillers of different types, chosen from the following types: oxides, sulfates and silicates, and preferably from titanium dioxide, an alkali metal or alkaline-earth metal sulfate and a silicate, and even more preferably from titanium dioxide, barium sulfate and tourmaline.

More preferably, the composition C comprises at least three mineral fillers of different types, chosen from the above types. Particularly preferably, the composition C comprises three mineral fillers M of different types which are an oxide, a sulfate and a silicate.

Even more preferably, the composition C comprises three mineral fillers M of different types selected from the group consisting of oxides, sulfates, carbonates, phosphates and silicates, at least one mineral filler being water insoluble. Preferably, at least two mineral filler are water insoluble.

Preference is given quite particularly to the titanium dioxide/alkaline-earth metal sulfate/silicate combination, and even more preferentially the titanium dioxide/barium sulfate/tourmaline combination.

In this particular case, the three mineral fillers M of different types are titanium dioxide, barium sulfate and tourmaline.

In this case, the respective weight proportions of the three mineral fillers (preferably titanium dioxide:barium sulfate:tourmaline) above are preferably between 80:10:10, 05:35:60, and 05:15:80 and more specifically these respective proportions are 13:35:52.

Preferably, the weight proportion of mineral fillers M relative to the total weight of the biodegradable polymeric composition is greater than or equal to 1 percent, preferably greater than or equal to 5 percent, even more preferably is greater than or equal to 30 percent.

Preferably, the weight proportion of the mineral fillers M relative to the total weight of the biodegradable polymeric composition is less than or equal to 60 percent, preferably is less than or equal to 50 percent, even more preferably is less than or equal to 40 percent.

The mineral filler(s) M according to the invention is (are) advantageously in the form of particles, which preferably have a diameter-average size, measured according to laser diffraction particle size analysis, of less than or equal to 10 micrometers, preferably less than or equal to 5 micrometers, even more preferably less than or equal to 2 micrometers. The laser diffraction particle size analysis can use, for example, Malvern or Cilas particle size analyzers.

One advantageous way to carry out the process consists in suspending the particles in water and in determining their particle size by laser diffraction using the method described in standard ISO 13320:2009.

It is preferable for the mineral fillers used in the present invention to have a particle size which is:
neither too small, so as to prevent any risk of the particles being able to leave the polymer matrix and introduce themselves into the human body through the skin or via the airways, or else disperse in the environment;
nor too large, which would make the incorporation of the particles into the polymer matrix more difficult and especially might make the cosmetic composition abrasive on contact with the skin, which might in the end have an irritant effect on the skin, for example in the case of particularly thin or sensitive skin.

Thus, the mineral filler(s) according to the invention is (are) in the form of particles, which advantageously have a diameter-average size, measured according to the laser diffraction particle size analysis method, ranging from 0.1 to 2 micron, more preferentially from 0.2 to 1.5 micron and even more preferentially from 0.2 to 1 micron.

The mineral fillers advantageously have a particle size distribution with 99 percent by volume of the particles having a size of less than 3.0 micron, preferably 90 percent by volume of the particles having a size of less than 1 micron. The particle size distribution is also measured by the abovementioned laser diffraction particle size analysis method (using, for example, Malvern or Cilas particle size analyzers).

The polymeric composition according to the invention preferably has more than 10 infrared radiation absorption peaks in the following ten frequency ranges: 3.00+/-0.30 micro m, 6.20+/-0.50 micro m, 8.00+/-0.25 micro m, 8.50+/-0.25 micro m, 9.00+/-0.25 micro m, 9.50+/-0.25 micro m, 10.00+/-0.25 micro m, 10.50+/-0.25 micro m, 11.00+/-0.25 micro m, 14.60+/-2.10 micro m, at least 1 peak being present in at least 7 of these ten frequency ranges.

The infrared radiation absorption spectrum can be determined by any method known to those skilled in the art. One possible method is the use of a Bruker Equinox 55 instrument, with a resolution of 4 cm⁻¹. In this case, the spectrum obtained is in ATR (“Attenuated Total Reflectance”) form, using a ZnSe crystal.

As has been set out above, the biodegradable polymeric composition can be in the form of particles or fibers.

When in the form of particles, said particles of biodegradable polymeric composition can have any shape and any size notably compatible with incorporation and dispersion in a carrier fluid in a cosmetic composition intended to be applied to the skin.

According to a first preferred embodiment of the invention, the particles of biodegradable polymeric composition have a substantially spherical shape, i.e. the particles have a shape similar to that of a sphere, which may be more or less regular, for example spheroids or ellipsoids and/or flattened.

In this embodiment, the particles of biodegradable polymeric composition advantageously have a diameter-average size of less than or equal to 800 micrometers, preferably less than or equal to 100 micrometers, even more preferably less than or equal to 60 micrometers.

The diameter-average size of the particles of biodegradable polymeric composition is measured according to the above mentioned laser diffraction particle size analysis method (using, for example, Malvern or Cilas particle size analyzers).

In this embodiment, the ratio between the diameter-average size of the particles of biodegradable polymeric composition and the diameter-average size of the mineral fillers M can also be optimized so as to avoid any risk of the particles being too small and being able to leave the biodegradable polymer matrix and introduce themselves into the human body or disperse in the environment, or, on the contrary, being too large, with the risk of making the composition abrasive on contact with the skin.

Thus, the ratio between the diameter-average size of the particles of biodegradable polymeric composition according to the invention and the diameter-average size of the mineral fillers M, these two sizes being measured according to the abovementioned laser diffraction particle size analysis method, is advantageously greater than or equal to 4. This ratio is preferably less than or equal to 3000. This ratio preferably ranges from 4 to 250, more preferentially from 4 to 100.

The particles of polymeric composition according to the invention can be prepared by the methods known to those skilled in the art for obtaining powders or fine particles of polymers, for example by milling, cryomilling or spray drying of the polymeric composition. Alternatively, the method described in patent application FR 2 899 591, the content of which is incorporated into the present application by way of reference, can be used.

According to this embodiment wherein the biodegradable polymeric composition is in the form of a particle, we can distinguish the value of the form factor required depending on the final cosmetic application. Indeed, for skin cleansing and exfoliation applications, the form factor is below 0.75 whereas for anti-aging application the form factor is preferably above 0.75. This form factor is measured according to method ASTM F1877-05.

According to a second preferred embodiment of the invention, the biodegradable polymer composition is in the shape of fibers.

The fibers can be in the form of filaments, staple fibers and yarns, which can then be transformed into fabrics such as knitted, woven and non-woven fabrics, and used in textile and/or industrial applications such as garments, footwear, fishing nets, cords, sewing threads, boats, and so forth.

For cosmetic application we can use short length fibers. In the case of short length fibers, the average length is preferably less than or equal to 100 mm, more preferentially less than or equal to 10 mm and even more preferentially less than or equal to 1.0 mm.

These fibers preferably have an equivalent average diameter ranging from 1 to 100 micrometers, preferably from 4 to 50 micrometers and more preferentially from 6 to 20 micrometers.

These two parameters (the average length and the equivalent average diameter of the fibers) are advantageously measured by optical microscopy.

In this second embodiment, the ratio between the size of the mineral filler(s) and the diameter of the fibers can also be optimized so as to avoid any risk of the particles being too small and being able to leave the polymer matrix and introduce themselves into the human body or disperse in the environment, or, on the contrary, being too large, with the risk of making the composition abrasive on contact with the skin.

Thus, the ratio between the equivalent average diameter of the fibers according to the invention and the diameter-average size of the mineral fillers, measured according to the above mentioned laser diffraction particle size analysis method, is then advantageously greater than or equal to 10. This ratio between the equivalent average diameter of the fibers and the diameter-average size of the mineral fillers is preferably less than or equal to 1000.

The fibers according to the invention can be prepared by methods known to those skilled in the art. The process can, for example, be carried out by melt spinning of the polymeric composition, so as to obtain filaments, which can then be cut up (by means of a guillotine device or any other means known to those skilled in the art) so as to obtain fibers having the desired length.

The present invention also includes a marine biodegradable polymeric composition comprising a composition C comprising at least one mineral fillers M of different types having properties of absorption and/or emission in the far infrared region ranging from wavelength of 2 micrometers to 20 micrometers, said composition C being dispersed in said biodegradable polymeric composition, wherein said biodegradable polymeric composition comprises at least one polymer.

According to one preferred embodiment, the mineral fillers M comprises at least three mineral fillers of different types having properties of absorption and/or emission in the far infrared region ranging from wavelength of 2 micrometers to 20 micrometers, two of them being selected from the group consisting of oxides, sulfates, carbonates and phosphates and the third one being a silicate.

In another preferred embodiment the polymer is selected from polyhydroxyalkanoate (PHA), polyamide (PA), polyglycolic acid (PGA), polycaprolactone (PCL) or polylactic acid (PLA), preferably a polyhydroxyalkanoate (PHA), more preferably a polyhydroxyalkanoate (PHA) selected in the group consisting of poly-3-hydroxybutyrate (PHB or P3HB), poly(3-hydroxypropionate) (PHP or P3HP), polyhydroxyvalerate (PHV), poly(hydroxybutyrate-hydroxyvalerate (PHBV), Poly(3-hydroxyhexanoate) (PHHx), copolymers thereof and blends thereof and in particular polyhydroxybutyrate (PHB), copolymers thereof and blends thereof.

When the above biodegradable polymeric composition is based on polylactic acid (PLA) or polyamide (preferably PA66, PA6, PA 5.6, PA6.10, PA10.10 and PA12), an additive A as described above in the description is preferably added.

Said specific composition can be in the form of particles or fibers, with the same meaning as the one state above in the description.

The subject of the present invention is finally the use of such a specific composition in cosmetic formulations or in industrial or textiles applications.

The marine biodegradable polymeric composition disclosed above can be used in cosmetic formulations.

According to this embodiment, the cosmetic formulation is a formulation for anti-aging, cleansing, sensorial modification, matifying, and moisturizing applications.

For this kind of application, particles of marine biodegradable polymeric composition according to the invention are advantageously used in the form of a dispersion in a cosmetic composition.

This dispersion is produced by dispersing said particles or fibers in a carrier fluid, i.e. a liquid medium which serves as a vehicle for said particles or fibers. This carrier fluid comprises water and/or one or more organic fluids.

According to the invention, the term “organic fluid” denotes organic liquids which can have very variable viscosities. Thus, the organic fluids usable in the invention can have a dynamic viscosity at 20° C.entigrade ranging from 10⁻⁴ to 10³ Pa·s, preferably from 0.5×10⁻³ to 10² Pa·s.

Such fluids can be water-miscible in any proportions. They can thus be chosen from monoalcohols containing from 2 to 4 carbon atoms, and polyols containing from 2 to 6 carbon atoms, such as, in particular, glycol, glycerol or sorbitol.

Such fluids can also be water-immiscible, and in this case, when the composition also contains water, said composition is then in the form of an emulsion. They can thus be chosen from natural or synthetic oils, in particular mineral oils, vegetable oils, fatty alcohols, fatty acids, esters containing at least one fatty acid and/or at least one fatty alcohol, and silicones.

The alcohols and acids mentioned above are those which contain from 8 to 32, preferably from 10 to 26 and more preferentially from 12 to 22 carbon atoms.

It is of course possible to use mixtures of organic fluids and in particular any mixtures of any of the fluids described above.

According to one particularly preferred embodiment, the carrier fluid contains water.

In this case, the cosmetic composition according to the invention advantageously contains at least 20 percent by weight of water, more preferentially at least 30 percent by weight of water and even more preferentially at least 50 percent by weight of water, relative to the total weight of said composition.

Likewise preferably, the cosmetic composition according to the invention contains, in addition to the water, one or more organic fluids.

In this case, the cosmetic composition according to the invention advantageously contains at least 5 percent by weight of organic fluid(s), more preferentially at least 10 percent by weight of organic fluid(s), relative to the total weight of said composition.

The cosmetic composition can also comprise all the conventional ingredients known to those skilled in the art as being part of the composition of cosmetic skin products. These ingredients can in particular, and in a nonlimiting way, be chosen from: thickeners, surfactants, moisturizing agents, skin conditioning agents, UV-screening agents, colored or noncolored pigments, antioxidants and preservatives.

The additional ingredients which can be used in the compositions according to the invention can in particular be chosen from those described in the International Cosmetic Ingredient Dictionary and Handbook, regularly published by The Cosmetic, Toiletry, and Fragrance Association.

According to one particularly advantageous embodiment, the cosmetic composition according to the invention also comprises one or more antiwrinkle active agents different from the mineral fillers according to the invention.

Such antiwrinkle active agents can in particular be chosen, in a nonlimiting manner, from:
retinoids, such as retinol, esters of a C2 to C22 acid and of retinol (for example, retinyl palmitate, retinyl acetate, retinyl propionate), retinal, retinoic acids;
natural or synthetic peptides, preferably those containing from 2 to 20 amino acids and/or amino acid derivatives, more preferentially from 2 to 10 amino acids and/or amino acid derivatives; the amino acid derivatives which may be present in oligopeptides are well known to those skilled in the art and include, inter alia, the isomers, esters and complexes, in particular metal complexes, of such amino acids;
alpha-hydroxy acids and beta-hydroxy acids (for example glycolic acid);
ketone acids (for example pyruvic acid);
hyaluronic acid, salts thereof (in particular sodium or potassium salts) and esters thereof.

The antiwrinkle active agents can be present in contents ranging from 0.01 percent to 10 percent by weight, preferably from 0.1 percent to 8 percent by weight and more preferentially from 0.5 percent to 5 percent by weight, relative to the total weight of the cosmetic composition of the invention.

The cosmetic composition according to the invention can be in very different forms, such as in particular, and in a nonlimiting way, liquids which are more or less viscous (such as fluids, milks or sera), lotions, more or less thick creams, pastes, gels, foams or sprays (sprayable compositions).

It can be a product intended essentially for skincare and/or for making up the skin (for example, a foundation, lipstick, face powder or eyeshadow composition).

According to one particularly preferred embodiment, the composition according to the invention is in the form of a cream, which preferably consists of an emulsion, and more preferentially of an oil-in-water emulsion.

The cosmetic composition according to the invention can be prepared by the methods known to those skilled in the art in the field of cosmetic product preparation. These methods generally comprise mixing the ingredients of the composition in one or more steps, and can also include heating and/or cooling steps.

The subject of the present invention is also a cosmetic treatment method for the skin, consisting in bringing the skin into contact with a biodegradable cosmetic composition as described above.

This method consists in particular in applying said cosmetic composition to the skin, on the area(s) to be treated. This application can be daily, twice daily (for example, morning and evening), or more episodic (every other day, once a week, etc).

It is thus a subject of the present invention to provide the use of such a cosmetic composition for preventing or reducing the signs of skin aging.

The subject of the present invention is finally the use of the marine biodegradable polymeric composition as described above in the form of fibers in industrial or textile applications.

The fibers can be in the form of filaments, staple fibers and yarns, which can then be transformed into fabrics such as knitted, woven and non-woven fabrics, and used in textile and/or industrial applications such as garments, footwear, fishing nets, cords, sewing threads, boats, and so forth.

The detailed description, given above, of the use according to the invention also applies to the method according to the invention.

### EXPERIMENTAL PART

### Examples

### Example 1

### 1.A – Materials

The materials used for the preparation of the samples are as follows:
✔ PHB with the following features:
   Origin: commercial: Biocycle®1000 sold by PHB Industrial S.A.
   Density (ASTM D792): 1.23 g/cm3
   Melt Flow Index (ASTM D1238): 15.0 g/10min
   Melting Point (ASTM D3418): 165-170° C.
   Izod Impact Resistance (ASTM D256): 20.4 J/m
   Modulus of Elasticity (ASTM D638): 3.07 GPa
   Elongation at break (ASTM D638): 2.24 %
   Tensile strength (ASTM D638): 32.4 MPa
✔ PHBV with the following features:
   Origin: commercial: ENMAT®Y1000 sold by TianAn
   Density (ASTM D792): 1.25 g/cm3
   Melt Flow Rate(190° C.- 2.16 kg) (ASTM1238): < 5.0 g/10min
   Melting Point (ASTM D3418): 177° C.
   Molecular weight: 450000
✔ PLGA with the following features:
   Origin: commercial: POLYLACTIC-CO-GLYCOLICACID (PLGA-50:50) sold by Nomisma Healthcare
   Molecular ratio of LA/GA by NMR: 1.0
   Viscosity: 0.25 dl/g
✔ Tourmaline
   Origin: commercial from Microservice
   Particle size (D50): 0.8 µm
✔ Barium sulfate
   Origin: commercial from Venator
   Particle size (D50): 0.8 µm
✔ Titanium dioxide
   Origin: commercial from Venator
   Diameter-average particle size of 0.3 µm
   Particle size D50: 0.8 µm
✔ Citric Acid
   Origin: commercial from Sigma Aldrich
   Role: thermal stabilizer additive
✔ Kaolin
   Origin: commercial from Ouro Branco
   Role: FIR additive
✔ Silica
   Origin: commercial from Ouro Branco
   Role: FIR additive

### 1.B Production of Polymeric Compositions According to the Invention

Polymeric compositions with the following content were produced according to below:
Polymeric composition A 69 wt% of PHB, and 31 wt% of additives:
   Tourmaline 15.5 wt%,
   Barium Sulfate 10.5 wt%,
   Titanium dioxide 4 wt% and
   Citric acid 1 wt%.
Polymeric composition B 84 wt% of PHB, and 16 wt% of additives:
   Tourmaline 7.75 wt%,
   Barium Sulfate 5.25 wt%,
   Titanium dioxide 2 wt% and
   Citric acid 1 wt%.
Polymeric composition C 69 wt% of PHB, and 31 wt% of additives:
   Silica 15 wt%,
   Kaolin 15 wt% and
   Citric acid 1 wt%.
Polymeric composition D 69 wt% of PHBV, and 31 wt% of additives:
   Tourmaline 15.5 wt%,
   Barium Sulfate 10.5 wt%,
   Titanium dioxide 4 wt% and
   Citric acid 1 wt%.
Polymeric composition E 70 wt% of PLGA, and 30 wt% of additives:
   Tourmaline 15 wt%,
   Barium Sulfate 10 wt%,
   Titanium dioxide 5 wt% and

The polymeric compositions are obtained according to the process described below.

### Polymer Drying Condition

The PHB and PHBV are dried in a Convection Drying Oven at 60° C. for 4 hours.

### Process Conditions

The materials of polymeric composition A were mixed and then extruded in a co-rotating twin-screw extruder coupled to a torque rheometer (Thermo Scientific™, model PolyLab™ OS Rheodrive 7/ Extruder HAAKE™ Rheomix OS PTW 16).

The mixture is processed in the twin-screw extruder according to the following conditions:

| Screw rpm (min⁻¹) | 60 |
|---|---|
| Feeding rate (%) | 11 |
| Temperatures (°C) | |
| Zone 1 | 166 |
| Zone 2 | 168 |
| Zone 3 | 169 |
| Zone 4 | 169 |
| Zone 5 | 170 |
| Zone 6 | 170 |

The extruder cylinder contains co-rotating screws that convey, mix and melt the polymer through 6 extruder heating zones with a gradient of temperature from 166 to 170° C., incorporating the additives to the melt polymer to produce the compound which is forced out for an extrusion die head. The compound is extruded in the form of molten strands, cooled in a water trough, pulled through a water stripper by pull rolls to a helical cutter of the pelletizer and then cut into pellets. Polymeric compositions B, C, and D were processed according to the same conditions of the polymeric composition A.

Polymeric composition E was processed in the Haake Reomix OS, with roller rotors and following conditions:
Speed: 40 rpm
Temperature: 110° C.

### 1.C Production of Particles of Polymeric Composition

The pellets of the polymeric composition obtained in the example 1B were grinded by Cryogenic grinding under the below conditions:
Equipment: Netzsch Fluidized Bed Jet Mill CGS10
Process condition:
Speed: 16000 rpm
Cryogenic fluid: liquid nitrogen

Particles of polymeric composition containing 69 wt% of PHB, 30 wt% of mineral fillers (tourmaline, barium sulfate and titanium dioxide) and 1% of citric acid were thus obtained, with particle size (D50) less than 19 micron, with density of 1.53 g/cm3 and shape factor of 0.86.

The particle size analysis was carried out by a laser diffraction particle size analyser (Mastersizer 2000. Malvern Instruments), the powder being dispersed in ethanol.

The density of the compound and the particle shape factor was measured according to ASTM D792 and ASTM F1877.

### 1.D - Marine Biodegradability Tests ASTM D6691-01(2017)

The particles of the compositions described in 1B with FIR emitting minerals according to the invention were obtained by grinding process described above in 1C, particles of virgin polymer (without FIR additives but same particle size, less than 850 micron, and produced according to same process described above) as comparative example and cellulose particles (without FIR additives but same particle size) as reference were tested according to ASTM D6691-01 (2017) standard method to measure their marine biodegradability. The results are summarized in the table 1 below.

TABLE 1

Absolute biodegradation of polymeric compositions prepared with FIR minerals and samples of virgin polymers.

| Sample | Description | Absolute biodegradation in seawater (%) |
|---|---|---|
| Laboratory Reference | cellulose | 65.2 |
| PHB | Virgin polymer | 20.2 |
| PHBV | Virgin polymer | 10.8 |
| PLGA | Virgin polymer | 23.5 |
| Composition A | PHB+30wt% of FIR1 minerals | 38.3 |
| Composition B | PHB+15wt% of FIR1 minerals | 25.5 |
| Composition C | PHB+30wt% of FIR2 minerals | 39.4 |
| Composition D | PHBV+30wt% of FIR1 minerals | 22.3 |
| Composition E | PLGA+30wt% of FIR1 minerals | 30.5 |

The absolute biodegradation results show that when polymeric compositions present FIR minerals the biodegradation of the polymers in marine environment is improved.

### Example 2

### 2.A — Materials

The materials used for the preparation of the samples are as follows:
✔ Polyamide 6.6 with the following features.
   Origin: commercial: POLYAMIDE 6.6 BRILLIANT produced by
   Rhodia Brasil S.A, Solvay Group.
   Density (ISO 845 ou 1183): 1.14 g/cm3
   Melting Point (ISO 11357): 265° C.
   Izod Impact Resistance (ISO180-2019):150 J/m
   Modulus of Elasticity (ISO 527-1:2012 ): 2.93 GPa
   Elongation at break (ISO 527-1:2012): 25%
   Tensile strength (ISO 527-1:2012): 65 MPa
✔ Tourmaline
   Origin: commercial from Microservice
   Particle size (D50): 0.8 µm
✔ Barium sulfate
   Origin: commercial from Venator
   Particle size D50: 0.8 µm
✔ Titanium dioxide
   Origin: commercial from Venator
   Particle size D50: 0.3 µm
✔ Polyethylene glycolpolymer, PEG35000, with molecular weight of 35000 g/mol
   Origin: Sigma Aldrich
✔ Ethoxylated/propoxylated block copolymer, Antarox L101
   Origin :Solvay

### 2.B Production of a Polymeric Composition According to the Invention

Polymeric compositions with the following content were produced according to below:
Composition F 95 wt% of PA66 and 5 wt% of additive
   Biosphere 201 5 wt%.
Composition G 65 wt% of PA66, and 35 wt% of additives:
   Tourmaline 15.5 wt%,
   Barium Sulfate 10.5 wt%,
   titanium dioxide 4 wt%
   Biosphere 201, 5 wt%

PA66 was dried in a Convection Drying Oven at 80° C. for 6 hours. The materials (PA66 and additives) were mixed and then extruded in a co-rotating twin-screw extruder SHJ20. The mixture (PA66 and additives) was processed in the twin-screw extruder according to the following conditions:

| Screw rpm (min⁻¹) | 460 |
|---|---|
| Feeding rate (%) | 10 |
| Temperatures (°C) | |
| Zone 1 | 271 |
| Zone 2 | 276 |
| Zone 3 | 281 |
| Zone 4 | 281 |
| Zone 5 | 284 |
| Zone 6 | 270 |

The extruder cylinder contains co-rotating screws that convey, mix and melt the polymer through 6 extruder heating zones with a gradient of temperature from 270 to 284° C., incorporating the additives to the melt polymer to produce the compound which is forced out for an extrusion die head.

The compound is extruded in the form of molten strands, cooled in a water trough, pulled through a water stripper by pull rolls to a helical cutter of the pelletizer and then cut into pellets.

### 2.C Production of Particles of Polymeric Composition

Equipment: Co-rotating twin-screw coupled to Thermo Scientific Torque Rheometer - model Polylab OS Rheodrive 7 / HAAKE Rheomex OS Extruder PTW16, L/D 16 mm.

Process condition:

The pellets produced as described in example 2.B were mixed with compatibilizing agent Antarox L101 (10 wt%) and PEG 35000 and processed in a twin-screw extruder (Co-rotating twin-screw Coupled to Thermo Scientific Torque Rheometer - model Polylab OS Rheodrive 7 / HAAKE Rheomex OS Extruder PTW16, L/D 16 mm). The temperature profile of the various zones during the process varied from 250° C. to 270° C. rotating at 250 rpm. The compound is extruded and cooled in water. Part of the compound is solubilized in water and the spherical particles are separated by sieving and dried.

Particles of polymeric composition containing 68 wt% of PA66, 30 wt% of mineral fillers (tourmaline, barium sulfate and titanium dioxide) and 2 wt% of Biosphere 201 were thus obtained, with particle size (D50) less than 28 micron, with density of 1.45 g/cm³ and shape factor of 0.98.

Particle size analysis was carried out by a laser diffraction particle size analyser (Mastersizer 2000. Malvern Instruments), the powder being dispersed in ethanol.

### 2.D - Marine Biodegradability Tests ASTM D6691-01(2017)

The particles of the compositions described in 2B with Biosphere 201 and FIR emitting minerals according to the invention were obtained by process described above in 2C, particles of virgin polymer (without FIR or Biosphere 201 additives but same particle size, less than 28 micron, and produced according to same process described above) as comparative example and cellulose particles (without FIR additives but same particle size) as reference were tested according to ASTM D6691-01 (2009) standard method to measure their marine biodegradability. The results are summarized in the table 2 below.

TABLE 2

Absolute biodegradation of polyamide compositions.

| Sample Description | Description | Absolute biodegradation in seawater (%) |
|---|---|---|
| Laboratory Reference | cellulose | 65.2 |
| Polyamide 66 | Virgin polymer | 2.8 |
| Composition F | PA66 + 5 wt% of Biosphere 201 | 22.6 |
| Composition G | PA66 + 5 wt% of Biosphere201+30 wt% of FIR minerals | 35.2 |

The absolute biodegradation showed that when polyamide additivated with Biospere 201 compositions present FIR minerals, the biodegradation of the polymers in marine environment is improved.

Therefore, surprisingly, it has been found that the use of the above claimed mineral fillers in a marine biodegradable polymeric composition allows an improved in the marine biodegradability of the resulting polymeric composition.

1. A method of improving marine biodegradability of a marine biodegradable polymeric composition comprising dispersing a composition C in the marine biodegradable polymeric composition, wherein the composition C comprises at least one mineral filler M having properties of absorption and/or emission in the far infrared region ranging from wavelength of 2 micrometers to 20 micrometers.

2. The method of claim 1, wherein the marine biodegradable polymeric composition contains a polymer selected from the group consisting of polyamides, polyesters, polysaccharides, polypeptides or proteins, cellulose and polymeric derivatives thereof, cellulose esters and polymeric derivatives thereof, copolymers thereof and blends thereof.

3. The method of claim 2, wherein the polymer is selected from the group consisting of polyhydroxyalkanoates (PHA), polymeric derivatives of cellulose, cellulose acetate polymers, polyglycolic acid, polycaprolactone, copolymers thereof and blends thereof.

4. The method of claim 3, wherein the polymer is a polyhydroxyalkanoate (PHA) selectedfrom the group consisting of poly-3-hydroxybutyrate (PHB or P3HB), poly(3-hydroxypropionate) (PHP or P3HP), polyhydroxyvalerate (PHV), poly(hydroxybutyrate-hydroxyvalerate (PHBV), Poly(3-hydroxyhexanoate) (PHHx), copolymers thereof and blends thereof.

5. The method of claim 1, wherein the marine biodegradable polymeric composition comprises:
(a) a polymer selected from the group consisting of polyamide (preferably PA66, PA6, PA 5.6, PA6.10, PA10.10 and PA12), polylactic acid (PLA), poly(butylene succinate) (PBS), poly(butylene adipate-co-terephthalate) (PBAT) and poly(vinyl acetate) copolymers and blends thereof, and
(b) an additive A being a composition comprising :
(i) at least one carbohydrate-based or starch-based or aromatic-ester modified polymeric material,
(ii) optionally a plasticizer, and
(iii) optionally water.

6. (canceled)

7. The method of claim 1, wherein the at least one mineral filler M is selected from the group consisting of oxides, sulfates, carbonates, phosphates and silicates.

8. (canceled)

9. The method of claim 7, wherein the composition C comprises at least two mineral fillers M which are different selected from the group consisting of oxides, sulfates, carbonates, phosphates and silicates, at least one mineral filler being a silicate.

10. The method of claim 9, wherein the composition C comprises three mineral fillers M of different types selected from the group consisting of oxides, sulfates, carbonates, phosphates and silicates, at least one mineral filler being a silicate.

11. (canceled)

12. The method of claim 11, wherein the three mineral fillers M of different types are titanium dioxide, barium sulfate and tourmaline.

13. The method of claim 1, wherein the at least one mineral filler M is in the form of particles that have a diameter-average size, measured according to laser diffraction particle size analysis, of less than or equal to 10 micrometers.

14. The method of claim 1, wherein the weight proportion of mineral fillers M relative to the total weight of the marine biodegradable polymeric composition is greater than or equal to 1 percent.

15. The method of claim 1, wherein the weight proportion of the mineral fillers M relative to the total weight of the marine biodegradable polymeric composition is less than or equal to 60 percent.

16. The method of claim 1, wherein the marine biodegradable polymeric composition is in the form of particles or fibers.

17. The method of claim 16, wherein the marine biodegradable polymeric composition is in the form of particles that have a diameter-average size, measured according to laser diffraction particle size analysis, of less than or equal to 800 micrometers.

18. The method of claim 16, wherein the marine biodegradable polymeric composition is in the form of particles that have a substantially spherical shape.

19. (canceled)

20. A marine biodegradable polymeric composition comprising a composition C comprising at least one mineral fillers M of different types having properties of absorption and/or emission in the far infrared region ranging from wavelength of 2 micrometers to 20 micrometers, said composition C being dispersed in said biodegradable polymeric composition, wherein said biodegradable polymeric composition comprises at least one polymer.

21. The marine biodegradable polymeric composition as claimed in claim 20, wherein the mineral fillers M comprises at least three mineral fillers of different types having properties of absorption and/or emission in the far infrared region ranging from wavelength of 2 micrometers to 20 micrometers, two of them being selected from the group consisting of oxides, sulfates, carbonates and phosphates and the third one being a silicate.

22. The marine biodegradable polymeric composition as claimed in claim 20, wherein the polymer is selected from polyhydroxyalkanoate (PHA), polyamide (PA), polyglycolic acid (PGA), polycaprolactone (PCL) or polylactic acid (PLA) copolymers thereof and blends thereof.

23. The marine biodegradable polymeric composition as claimed in claim 20, wherein the marine biodegradable polymeric composition is in the form of particles or fibers.

24. (canceled)

25. (canceled)

26. (canceled)
